(19) United States
(12) Patent Application Publication
(10) Pub. No.: US 2022/0233772 A1
(43) Pub. Date: Jul. 28, 2022

(54) SYSTEMS AND METHODS FOR AUTOMATED INSULIN DELIVERY FOR DIABETES THERAPY

(71) Applicant: Tandem Diabetes Care, Inc., San Diego, CA (US)

(72) Inventors: Thomas R. Ulrich, Oceanside, CA (US); Michael Michaud, San Diego, CA (US)

(21) Appl. No.: 17/587,434

(22) Filed: Jan. 28, 2022

Related U.S. Application Data
(60) Provisional application No. 63/142,813, filed on Jan. 28, 2021.

(51) Int. Cl. A61M 5/172; G16H 20/17

(52) U.S. Cl. CPC A61M 5/1723; A61M 2205/3561; A61M 2230/201; G16H 20/17

(57) ABSTRACT

Disclosed herein are systems and methods for automated insulin delivery that aid in treatment when a user has eaten and has forgotten to deliver a meal bolus to counteract the carbohydrates consumed in the meal. The systems and methods disclosed herein can modify a limiter function that limits an amount by which a basal rate can be increased following a given glucose level reading in certain circumstances. For example, if the user's current or predicted future glucose level is high and the user's glucose level is increasing above a threshold rate, the limit can be increased from a standard limit. Such a glucose response may indicate that the user has eaten, but forgotten to instruct a meal bolus. By increasing the basal rate increase limit, the system can therefore react to and address the missed meal bolus.

### RELATED APPLICATION

This application claims the benefit of U.S. Provisional Application No. 63/142,813 filed Jan. 28, 2021, which is hereby fully incorporated herein by reference.

### FIELD OF THE INVENTION

The present invention relates generally to ambulatory infusion pumps and, more particularly, to operation of ambulatory infusion pumps in a closed-loop or semi-closed-loop fashion.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring (CGM) devices. A CGM device consists of a sensor placed under the patient's skin and affixed to the patient via an adhesive patch, a transmitter, and a monitor. A CGM device samples the patient's interstitial fluid periodically (e.g. once every 1-5 minutes) to estimate blood glucose levels over time. CGMs are advantageous because they provide more frequent insights into a user's blood glucose levels yet do not require a finger stick each time a reading is taken.

Ambulatory infusion pumps may incorporate a CGM within the infusion pump device or may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

In some cases, ambulatory insulin pumps may be configured to deliver insulin based on CGM data in a closed-loop or semi-closed-loop fashion. Some systems including these features may be referred to as automated insulin delivery (AID) systems or artificial pancreas systems because these systems serve to mimic biological functions of the pancreas for persons with diabetes. Some AID systems primarily deliver medicament automatically based on CGM readings, but also enable users to program meal boluses. Consumption of carbohydrates in a meal causes blood glucose to rise, which can be counteracted by insulin or other medicament delivered in a meal bolus.

### SUMMARY OF THE INVENTION

Disclosed herein are systems and methods for automated insulin delivery that aid in treatment when a user has eaten and has forgotten to deliver a meal bolus to counteract the carbohydrates consumed in the meal. The systems and methods disclosed herein can modify a limiter function that limits an amount by which a basal rate can be increased following a given glucose level reading in certain circumstances. For example, if the user's current or predicted future glucose level is high and the user's glucose level is increasing above a threshold rate, the limit can be increased from a standard limit. This is because a high glucose level, coupled with a high rate of increase warrants a more aggressive response to prevent hyperglycemia. Such a glucose response may indicate that the user has eaten, but forgotten to instruct a meal bolus. By increasing the basal rate increase limit, the system can therefore react to and address the missed meal bolus.

In an embodiment, a system for closed loop diabetes therapy can include a pump mechanism configured to facilitate delivery of insulin to a user, a communications interface adapted to receive glucose levels from a continuous glucose monitor, a user interface and at least one processor functionally linked to the pump mechanism, the user interface and the communications device. The processor can be configured to automatically modify a basal rate of insulin delivered to the user with a closed loop delivery algorithm based on glucose levels received from the continuous glucose monitor with the closed loop delivery algorithm configured to calculate modifications to the basal rate to maintain the user's glucose levels at a glucose target and include a limiter function providing a limit on an amount by which the basal rate can be increased from a previous basal rate. If it is determined based on the glucose levels received from the continuous glucose monitor that the user may have eaten a meal and not taken a bolus of insulin to counteract the carbohydrates consumed in the meal, the amount by which the limiter function will permit the basal rate to be increased from the previous basal rate can be increased. A modified basal rate can then be determined according to the increased amount insulin automatically delivered to the user with the pump mechanism according to the modified basal rate.

In an embodiment, a system for closed loop diabetes therapy can include a pump mechanism configured to facilitate delivery of insulin to a user, a communications interface adapted to receive glucose levels from a continuous glucose monitor, a user interface and at least one processor functionally linked to the pump mechanism, the user interface and the communications device. The processor can be configured to automatically modify a basal rate of insulin delivered to the user with a closed loop delivery algorithm based on glucose levels received from the continuous glucose monitor with the closed loop delivery algorithm configured to calculate modifications to the basal rate to maintain the user's glucose levels at a glucose target and include a limiter function providing a limit on an amount by which the basal rate can be increased from a previous basal rate. If it is determined based on the glucose levels received from the continuous glucose monitor that that a glucose level of the user is above the glucose target by more than a predetermined amount and that the user's glucose levels are increasing at greater than a predetermined rate, the amount by which the limiter function will permit the basal rate to be increased from the previous basal rate can be increased. A modified basal rate can then be determined according to the increased amount insulin automatically delivered to the user with the pump mechanism according to the modified basal rate.

In an embodiment, a system for closed loop diabetes therapy can include a pump mechanism configured to facilitate delivery of insulin to a user, a communications interface adapted to receive glucose levels from a continuous glucose monitor, a user interface and at least one processor functionally linked to the pump mechanism, the user interface and the communications device. The processor can be configured to automatically modify a basal rate of insulin delivered to the user with a closed loop delivery algorithm based on glucose levels received from the continuous glucose monitor with the closed loop delivery algorithm configured to calculate modifications to the basal rate to maintain the user's glucose levels at a glucose target and include a limiter function providing a limit on an amount by which the basal rate can be increased from a previous basal rate. If it is determined based on the glucose levels received from the continuous glucose monitor that that a current glucose level of the user is over a high glucose threshold and a predicted future glucose level of the user is a predetermined amount over the glucose target, the amount by which the limiter function will permit the basal rate to be increased from the previous basal rate can be increased. A modified basal rate can then be determined according to the increased amount insulin automatically delivered to the user with the pump mechanism according to the modified basal rate.

### BRIEF DESCRIPTION OF THE DRAWINGS

The invention may be more completely understood in consideration of the following detailed description of various embodiments of the invention in connection with the accompanying drawings, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 is an embodiment of a CGM for use with embodiments of the disclosure.

FIG. 5 is a schematic representation of a closed-loop insulin delivery algorithm according to the disclosure.

FIG. 6 is a graphical representation of a limiter function for an automated insulin delivery system according to the disclosure.

FIG. 7 is a graphical representation of a limiter function for an automated insulin delivery system according to the disclosure.

FIG. 8 is a graphical representation of a limiter function for an automated insulin delivery system according to the disclosure.

FIG. 9 is a flowchart of steps for a method of diabetes therapy according to the disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

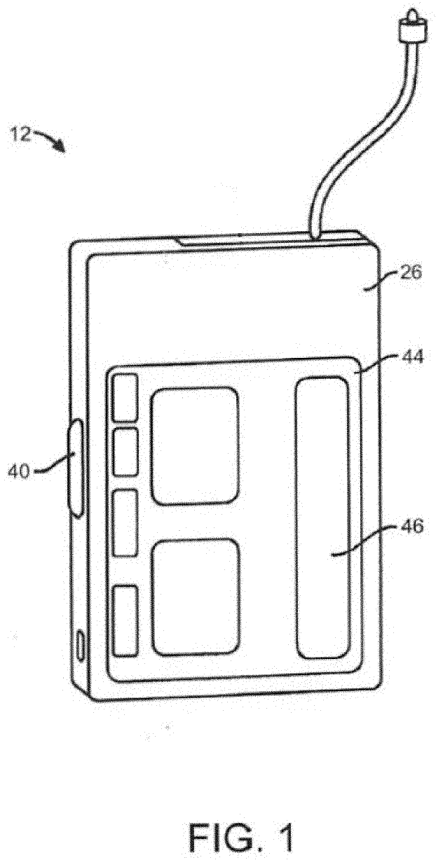

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

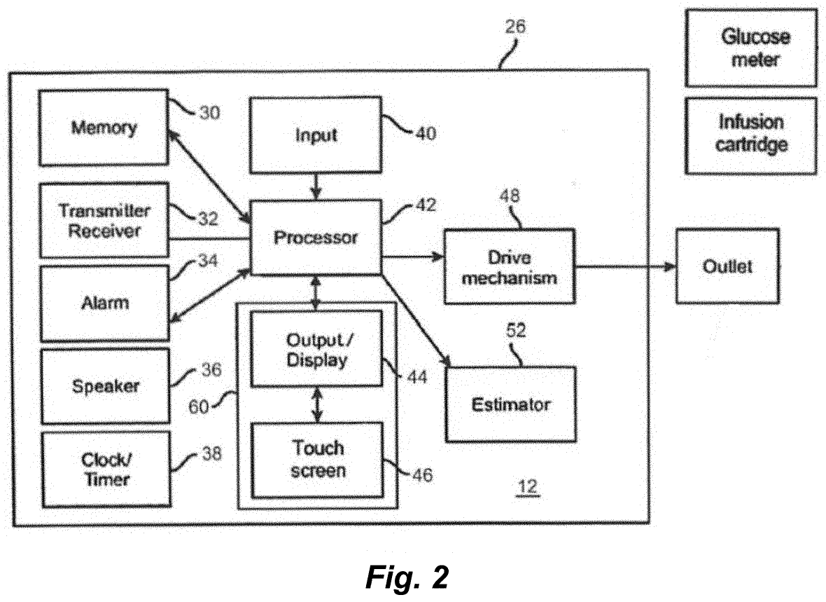

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device (e.g., laptop computer, personal computer, tablet computer, smartphone, electronic watch, electronic health or fitness monitor, or personal digital assistant). In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, or the like.

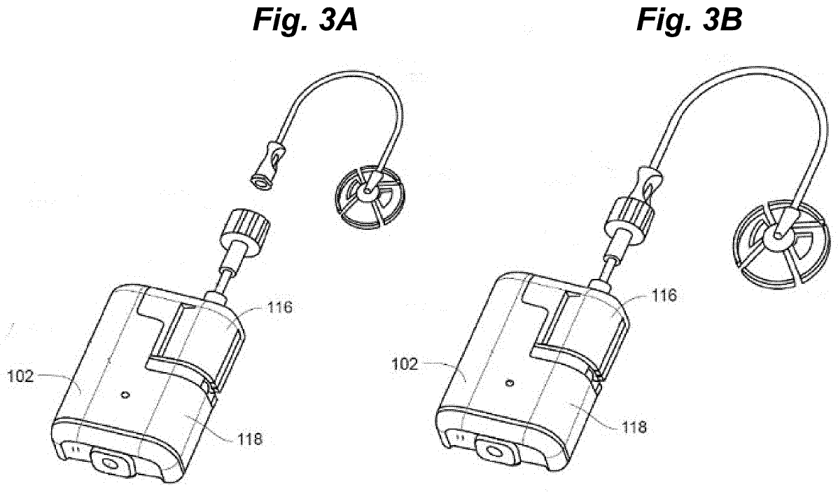

FIGS. 3A-3B depicts a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receive commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

In some embodiments, all elements of an infusion pump system such as, e.g., the user interface, processor(s), pump mechanism, etc., reside in a single device, such as an infusion pump. In other embodiments, an infusion pump system may be a distributed system in which portions of the functionality such as, e.g., the user interface, speaker, processor, dosing algorithm, etc. may reside in separate devices such as in the infusion pump, dedicated remote control and/or other mobile device such as a mobile phone, or central computer system such as a cloud computing system.

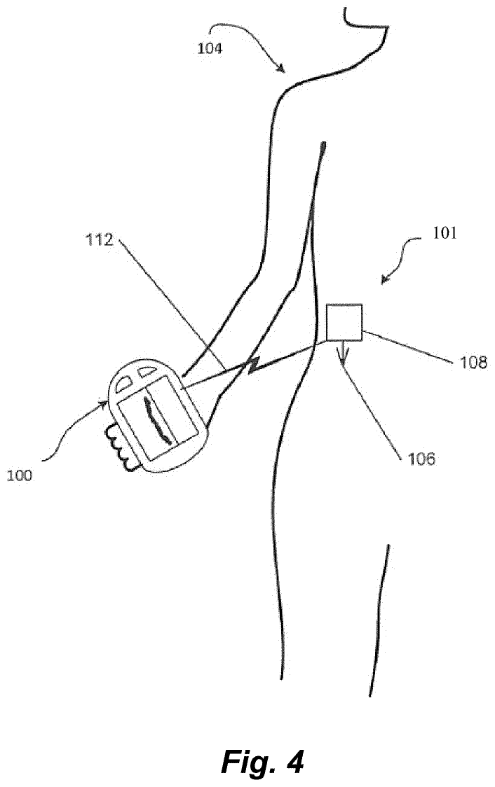

FIG. 4 depicts an example CGM system that can be used in conjunction with one or more embodiments of the ambulatory infusion pump system of the present disclosure. The CGM system includes a sensor 101, a sensor probe 106, a sensor body 108, a receiver, and a monitor (receiver and monitor are depicted as device 100 in FIG. 4). The sensor 101 is removably affixed to a user 104 and includes a sensor probe 106 configured for transcutaneous insertion into the user 104. When placed, the sensor probe 106 reacts with the user's interstitial fluid which produces a signal that can be associated with the user's blood glucose level. The sensor 101 further includes a sensor body 108 that transmits data associated with the signal to the receiver 100 via wired or wireless connection (as represented by arrow line 112). In preferred embodiments, the receiver 100 receives the transmitted data wirelessly by any suitable means of wireless communication. By way of example only, this wireless communication may include a near field communication (NFC) radio frequency (RF) transmitter or a transmitter operating according to a “Wi-Fi” or Bluetooth® protocol, Bluetooth® low energy protocol or the like. Further detail regarding such systems and definitions of related terms can be found in, e.g., U.S. Pat. Nos. 8,311,749, 7,711,402 and 7,497,827, each of which is hereby incorporated by reference in its entirety.

With the infusion pump and CGM interfaced, the CGM can automatically transmit the CGM data to the pump. The pump can then use this data to automatically determine therapy parameters and suggest a therapy adjustment to the user or automatically deliver the therapy adjustment to the user. These therapy parameters including thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

For example, if the CGM readings indicate that the user has or is predicted to have a high blood glucose level, the ambulatory infusion system can automatically calculate an insulin dose sufficient to reduce the user's blood glucose level below a threshold level or to a target level and automatically deliver the dose. Alternatively, the ambulatory infusion system can automatically suggest a change in therapy upon receiving the CGM readings such as an increased insulin basal rate or delivery of a bolus, but can require the user to accept the suggested change prior to delivery rather than automatically delivering the therapy adjustments.

By way of further example, if the CGM readings indicate that the user has or is predicted to have a low blood glucose level (hypoglycemia), the ambulatory infusion system can, for example, automatically reduce or suspend a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the patient address the hypoglycemic condition as necessary (e.g., ingest carbohydrates), singly or in any desired combination or sequence.

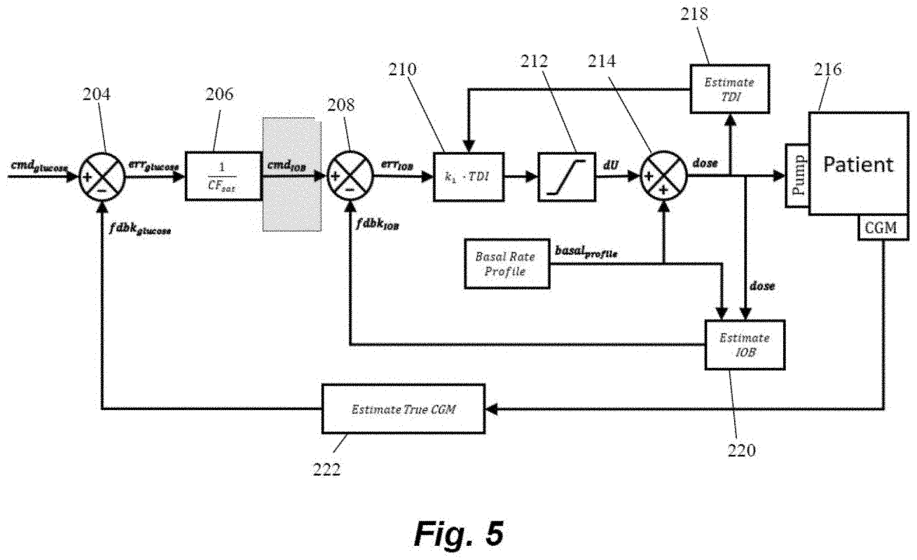

A schematic representation of a control algorithm for automatically adjusting insulin delivery based on CGM data is depicted in FIG. 5. This figure depicts an algorithm for increasing basal rate that utilizes a cascaded loop. The logic for decreasing basal rate is not depicted. In the depicted embodiment, there is a glucose set-point/command (cmd) that is determined at step 202. The glucose set point is a target value at which the algorithm attempts to maintain a user's blood glucose. This value can vary based on a number of factors, including the user's physiology, whether the user is awake or asleep, how long the user has been awake, etc. The glucose set point is compared to the actual CGM feedback (fdbk) at step 204 to determine a glucose error value (err) that is the difference between the set point and the feedback. In various embodiments, the CGM feedback can be a current glucose level reading received from a CGM or can be a predicted future glucose value based on previous glucose readings. For example, the system may predict a glucose level 30 minutes in the future (Gpred30) and utilized the predicted value as the fdbk glucose value. The errGLUCOSE value at step 206 is multiplied by a constant (1/CF), in which CF is the user's correction factor, or amount by which one unit of insulin lowers the user's blood glucose. This calculation determines how much insulin is needed to correct the glucose error, which is how much insulin on board (IOB) is needed in the user's body. This IOB value then determines an appropriate estimated insulin on board (IOB) set point for the patient.

The estimated IOB level determined at step 206 is then taken as the command (cmdIOB) for the inner loop and based on a difference of an IOB feedback value (fdbkIOB) and the cmdIOB set point at step 208, an IOB error value (errIOB) is determined. At step 210, the errIOB value is multiplied by a constant kl (relating to insulin-dependent glucose uptake in the body) and an estimate of the total daily insulin (TDI) of the user. This adjusts the errIOB to be proportional to the constant and the user's total daily intake of insulin. At step 212, a limiter function is applied to the value calculated at step 210. As will be discussed in further detail below, the limiter function can prevent the calculated amount from being larger or smaller than preset limits. The result is an insulin amount dU, which is the amount by which the user's stored basal rate should be modified. The insulin delivery rate for the user for the next closed loop interval is therefore calculated by modifying the user's stored basal rate profile by the dU value at step 214.

After the dose is calculated, it can be delivered to the user at step 216 and can also be used to update the estimated TDI for the user at step 218. The dose can also be used to update the estimated IOB level for the user at step 220 by comparing the actual insulin delivered to the programmed basal rate. The updated estimated IOB then becomes the new fdbkIOB for the IOB comparison at step 208. When new CGM values are received from the CGM, an estimated true CGM can be determined based on various factors such as, for example, the calibration status of the CGM sensor. The estimated true CGM value then becomes the new fdbkGLUCOSE value for the outer loop comparison with cmdGLUCOSE at step 204 or the estimated true CGM value can be used to update the predicted future glucose level (i.e., Gpred30) for the comparison. The algorithm then proceeds through to calculate a new estimated IOB and to the inner IOB loop for calculation of an insulin dose as described above. In one embodiment, a new CGM value is received every 5 minutes and therefore the algorithm executes as set forth above every 5 minutes.

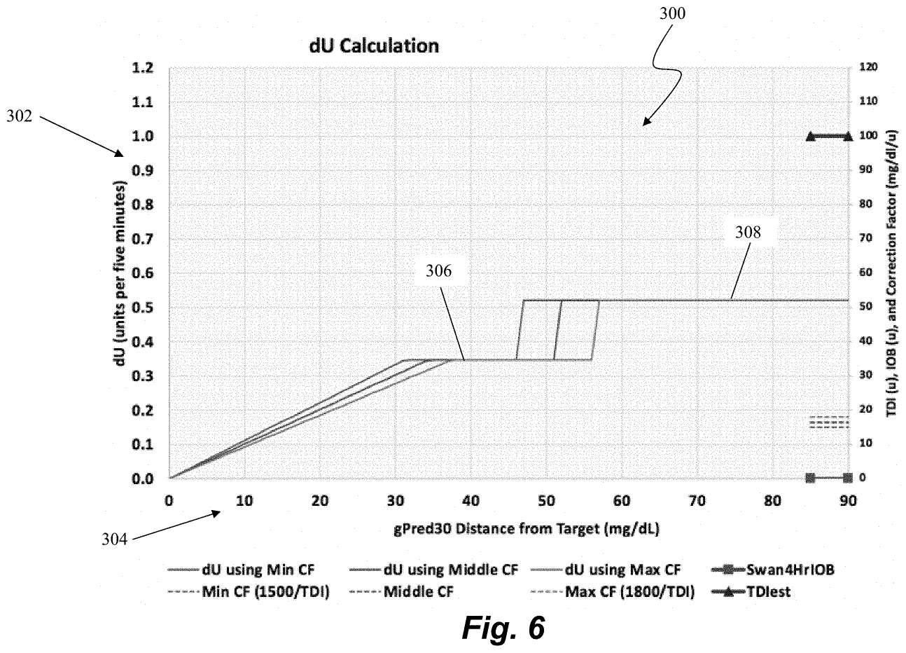

Referring now to FIG. 6, a graphical representation 300 of the limiter function applied at step 212 of FIG. 5 is depicted. The graph depicts the amount by which the basal rate is modified (dU) 302 each interval based on a distance 304 that the glucose feedback (e.g., gPred30) is from a target value. The graph depicts plots for three different calculations that can be used depending on a desired method of determining a correction factor for the user (e.g., 1500/TDI, 1800/TDI or an average of the two). For each graph, it can be seen that dU increases as the distance from target increases until it reaches a first limit 306. However, the first limit 306 can be increased up to a second higher limit 308 in certain circumstances. For example, if the gPred30 value is both over a certain amount from the target and the current glucose estimate is over a certain high threshold, then the limit on the dU increase can be increased to the second limit. The second higher limit can therefore be employed when not only is it determined that the user's glucose level will be above the target in 30 minutes, but also the user is already currently over a high glucose threshold. For example, in one embodiment the glucose target value may be 112.5 mg/dL and the high glucose threshold may be 180 mg/dL. If the user is both predicted to be more than, e.g., 50 mg/dL over the 112.5 mg/dL target in 30 minutes and is currently over the 180 mg/dL high glucose threshold, the higher limit can be employed to more quickly bring the user's glucose levels down to the target value. By utilizing both a current and predicted future value prior to employing the greater limit, the system reduces the risk of an increased dose being too large and causing the user's glucose level to go too low in response to the increased insulin dose.

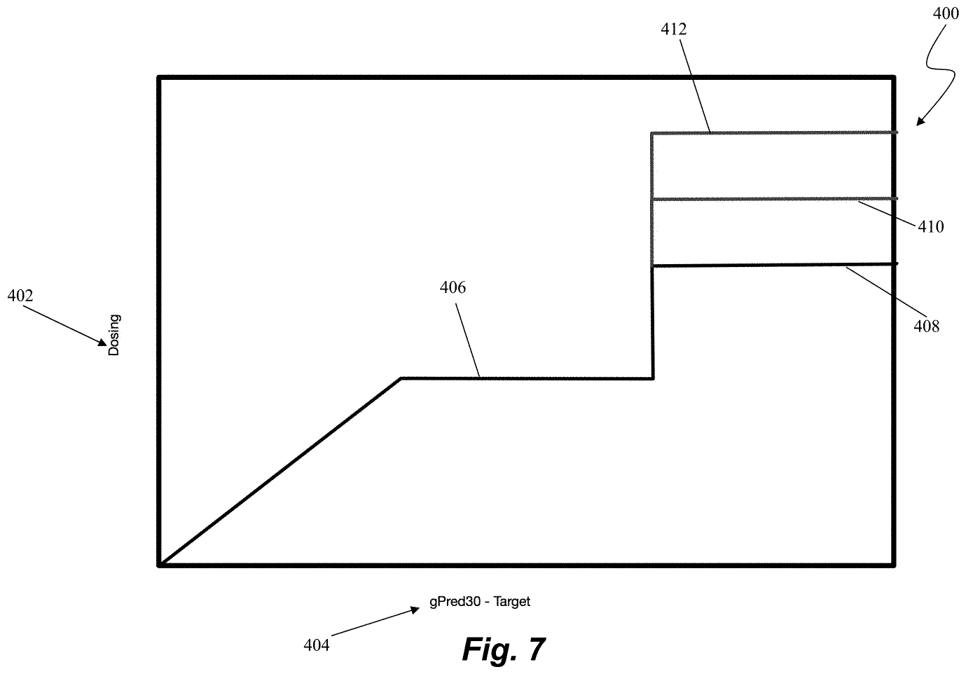

Referring now to FIG. 7, a graphical representation 400 of the limiter function applied at step 212 of FIG. 5 is depicted according to another embodiment. This figure is simplified to show only a single plot, but it should be understood that the figure could employ multiple plots based on different correction factor methods as depicted in FIG. 6. This embodiment similarly depicts an increasing dU 402 as the distance from target 404 increases up to a first limit 406. In this example, an additional consideration of a rate of change of the glucose level of the user is taken to account. If gPred30 and/or a current glucose level is above a high threshold (e.g., 180 mg/dL) and the user's glucose level is increasing at a rate above a high threshold, a higher limit 408 can be applied that supersedes and is greater than limit 308 in FIG. 6. In embodiments, the increasing rate of the user's glucose levels can be determined based off of a current glucose level estimate (gEst) and a predicted future glucose level (e.g., gPred30) because to the extent the predicted future glucose level is greater than the current glucose level estimate, the user's glucose levels are increasing. For example, the limit 408 can be applied if gPred30−gEst>60 mg/dL, which would equate to a change of 2 mg/dL per minute over the 30 minute difference between gEst and gPred30, and if the user's current and/or predicted future glucose level is over, e.g., 180 mg/dL. This is because a high glucose level, coupled with a high rate of increase, warrants a more aggressive response to prevent hyperglycemia. Such a glucose response may indicate that the user has eaten, but forgotten to instruct a meal bolus. By increasing the limit on dU, the system can therefore react to and address the missed meal bolus. In various embodiments, the difference between gEst and gPred30 that can trigger use of the higher limit can be, e.g., between about 10 mg/dL and 90 mg/dL.

In addition, to providing a higher limit 408 for dU in circumstances where the glucose level rate of change exceeds a threshold, the system can include additional stepped or tiered limits depending on the degree to which the user's glucose levels are over the threshold and/or increasing above the threshold rate. For example, the initial higher limit 308 may be utilized when the current or predicted glucose level is 180-190 mg/dL, the next higher limit 310 at 190-200 mg/dL and the highest limit 312 at over 200 mg/dL. In other embodiments, narrower or wider glucose level ranges can be utilized to separate the tiers, such as, for example, 180-195 mg/dL for limit 308, 195-210 mg/dL for limit 310 and above 210 mg/dL for limit 312. Alternatively or additionally, the higher tiers can be employed based on how high above the threshold rate of change the current glucose rate of change has been measured. It should be understood that although three steps or tiers are depicted, that any number of tiers could be utilized. The use of such tiers further protects against hyperglycemia by allowing more aggressive basal rate increases when the user has higher glucose levels.

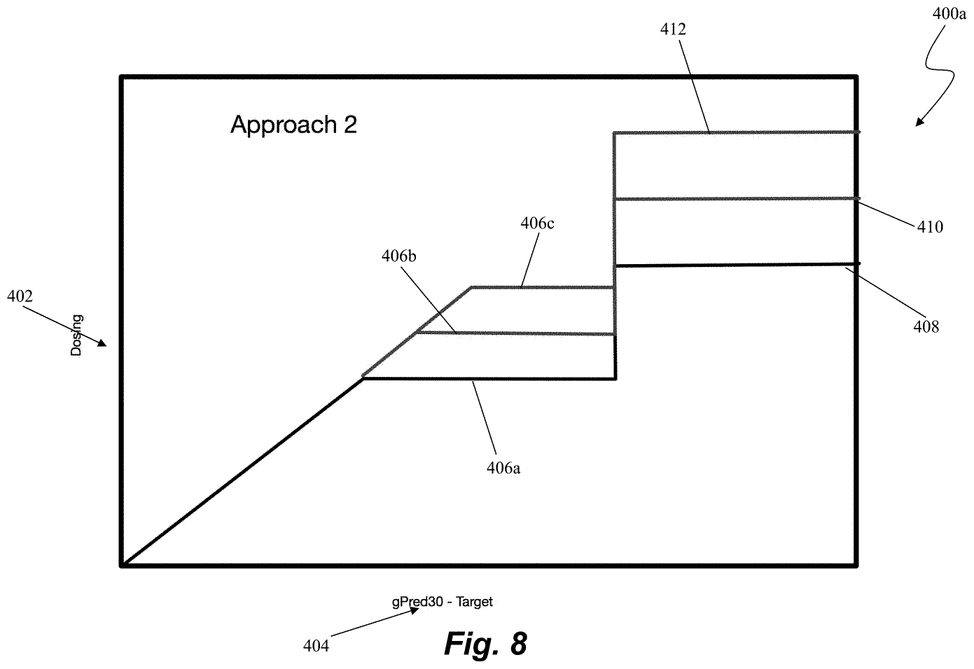

FIG. 8 depicts a similar approach 400a for limiter function 212 that employs a tiered or stepped approach to both limit 406 and limit 410. First limit 406 in this embodiment therefore includes a first tier 406a, a second tier 406b and a third tier 406c. Similar to first limit 306, the first limit 406a-406c can be implemented after dU rises the further the current or predicted glucose level is from the target to limit a maximum basal rate increase for a distance from target for a given reading. The tiered limits can then additionally be employed based on a current glucose level reading. For example, first limit 406a may be employed if the user's current or predicted glucose level is below 180 mg/dL and additional tiered limits 406b, 406c employed at steps based on how far the user's current or predicted glucose level is above 180 mg/dL. First limits 406b and 406c therefore mimic the second limit 308 in limiter function 300 by being employed only when the user's glucose level is a threshold amount above a target value and a high threshold, such that limiter function 400a essentially functions to combine tiered higher limits 308 of limiter function 300 and 408-412 of limiter function 400.

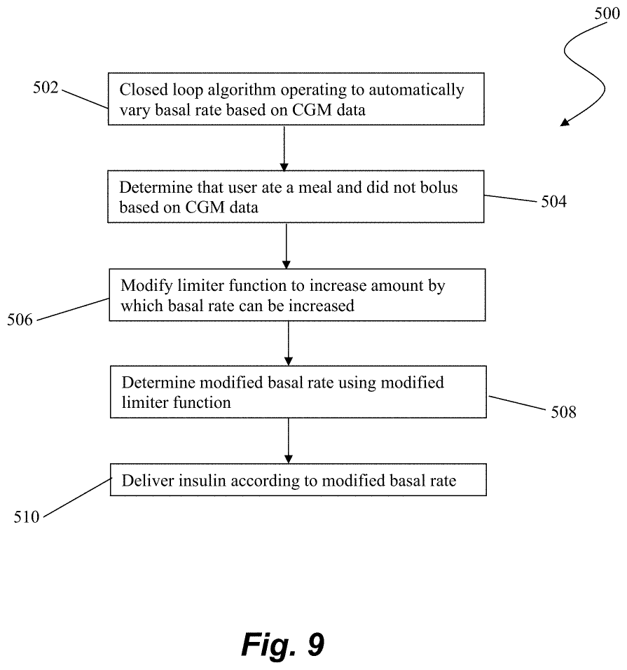

FIG. 9 depicts a flowchart of steps for a method 500 of diabetes therapy according to the disclosure. At step 502, the system is executing a closed loop algorithm that automatically modifies a basal rate of a user based on glucose levels received from a continuous glucose monitor to maintain the user's glucose levels at a glucose target. At step 504, it is determined from the user's glucose levels that a user may have eaten a meal but did not deliver a meal bolus to counteract the carbohydrates in the meal based. This determination can be made according to any of the embodiments described herein. For example, this determination can be made if both the user's glucose level is currently over a high threshold (e.g., 180 mg/dL) and the user's glucose level is predicted to be a predetermined amount greater than a glucose target level (e.g., 50 mg/dL over a 112.5 mg/dL target). Alternatively or additionally, such a determination can be made if a current and/or future glucose level of the user is above a high threshold (e.g., 180 mg/dL) and the user's glucose level is increased at a rate at or above a threshold rate (i.e., 2 mg/dL per minute). In response to determining that the user may have eaten a meal and not bolused, at step 506, the system can modify a limiter function in the closed loop algorithm to increase the amount by which the basal rate can be increased from a previous basal rate. A modified basal rate can be determined with the closed loop algorithm employing the modified limiter function at step 508 and automatically delivered to the user at step 510. As noted above, modifications to the basal rate can be calculated at regular, fixed intervals (e.g., every 5 minutes) such that the modified basal rate can be delivered to the user for the subsequent interval (i.e., the next 5 minutes) and then the method is repeated based on updated glucose level data at the next interval to determine whether the standard limiter function should be resumed or the modified limiter function can continue to be employed.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10/541,987; 10,569,016; 10,736,037; 10,888,655; 10,994,077; 11,116,901; and 11,224,693 and commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0206420; 2020/0261649; 2020/0306445; 2020/0329433; 2020/0368430; 2020/0372995; 2021/0001044; 2021/0113766; 2021/0154405; and 2021/0353857 and commonly owned U.S. patent application Ser. Nos. 17/368,968; 17/459,129; 17/517,885 and 17/573,705.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. A system for closed loop diabetes therapy, comprising:
a pump mechanism configured to facilitate delivery of insulin to a user;
a communications interface adapted to receive glucose levels from a continuous glucose monitor;
at least one processor functionally linked to the pump mechanism and the communications device, the at least one processor configured to:
automatically modify a basal rate of insulin delivered to the user with a closed loop delivery algorithm based on glucose levels received from the continuous glucose monitor, the closed loop delivery algorithm configured to calculate modifications to the basal rate to maintain the user's glucose levels at a glucose target, the closed loop delivery algorithm including a limiter function providing a limit on an amount by which the basal rate can be increased from a previous basal rate;
determine based on the glucose levels received from the continuous glucose monitor that the user may have eaten a meal and not taken a bolus of insulin to counteract the carbohydrates consumed in the meal;
increase the amount by which the limiter function will permit the basal rate to be increased from the previous basal rate in response to determining that the user may have eaten a meal and not taken a bolus of insulin to counteract the carbohydrates consumed in the meal;
determine a modified basal rate according to the increased amount; and
automatically deliver insulin to the user with the pump mechanism according to the modified basal rate.

2. The system of claim 1, wherein the at least one processor is configured to determine the user may have eaten a meal and not taken a bolus of insulin to counteract the carbohydrates consumed in the meal when a current glucose level of the user is over a high glucose threshold and a predicted future glucose level of the user is a predetermined amount over the glucose target.

3. The system of claim 1, wherein the at least one processor is configured to determine the user may have eaten a meal and not taken a bolus of insulin to counteract the carbohydrates consumed in the meal when a glucose level of the user is above the glucose target by more than a predetermined amount and the user's glucose levels are increasing at greater than a predetermined rate.

4. The system of claim 3, wherein the glucose level of the user is a current glucose level of the user received from the continuous glucose monitor.

5. The system of claim 3, wherein the glucose level of the user is a predicted future glucose level of the user based on the glucose levels received from the continuous glucose monitor.

6. The system of claim 3, wherein determining that a user's glucose levels are increasing at greater than a predetermined rate includes comparing a predicted future glucose level of the user and a current glucose level of the user to a threshold.

7. The system of claim 1, wherein the glucose target used by the closed loop algorithm is a single glucose value.

8. The system of claim 1, wherein the glucose target used by the closed loop algorithm is a target glucose range.

9. A system for closed loop diabetes therapy, comprising:
a pump mechanism configured to facilitate delivery of insulin to a user;
a communications interface adapted to receive glucose levels from a continuous glucose monitor;
at least one processor functionally linked to the pump mechanism and the communications device, the at least one processor configured to:
automatically modify a basal rate of insulin delivered to the user with a closed loop delivery algorithm based on glucose levels received from the continuous glucose monitor, the closed loop delivery algorithm configured to calculate modifications to the basal rate to maintain the user's glucose levels at a glucose target, the closed loop delivery algorithm including a limiter function providing a limit on an amount by which the basal rate can be increased from a previous basal rate;
determine from the glucose levels received from the continuous glucose monitor that a glucose level of the user is above the glucose target by more than a predetermined amount and that the user's glucose levels are increasing at greater than a predetermined rate;
increase the amount by which the limiter function will permit the basal rate to be increased from the previous basal rate in response to determining that the glucose level of the user is above the glucose target by more than the predetermined amount and that the user's glucose levels are increasing at greater than the predetermined rate;
determine a modified basal rate according to the increased amount; and
automatically deliver insulin to the user with the pump mechanism according to the modified basal rate.

10. The system of claim 9, wherein the glucose level of the user is a current glucose level of the user received from the continuous glucose monitor.

11. The system of claim 9, wherein the glucose level of the user is a predicted future glucose level of the user based on the glucose levels received from the continuous glucose monitor.

12. The system of claim 9, wherein determining that a user's glucose levels are increasing at greater than a predetermined rate includes comparing a predicted future glucose level of the user and a current glucose level of the user to a threshold.

13. The system of claim 9, wherein the glucose target used by the closed loop algorithm is a single glucose value.

14. The system of claim 9, wherein the glucose target used by the closed loop algorithm is a target glucose range.

15. A system for closed loop diabetes therapy, comprising:
a pump mechanism configured to facilitate delivery of insulin to a user;
a communications interface adapted to receive glucose levels from a continuous glucose monitor;
at least one processor functionally linked to the pump mechanism and the communications device, the at least one processor configured to:
automatically modify a basal rate of insulin delivered to the user with a closed loop delivery algorithm based on glucose levels received from the continuous glucose monitor, the closed loop delivery algorithm configured to calculate modifications to the basal rate to maintain the user's glucose levels at a glucose target, the closed loop delivery algorithm including a limiter function providing a limit on an amount by which the basal rate can be increased from a previous basal rate;
determine based on the glucose levels received from the continuous glucose monitor that a current glucose level of the user is over a high glucose threshold and a predicted future glucose level of the user is a predetermined amount over the glucose target;
increase the amount by which the limiter function will permit the basal rate to be increased from the previous basal rate in response to determining that the current glucose level of the user is over a high glucose threshold and the predicted future glucose level of the user is a predetermined amount over the glucose target;
determine a modified basal rate according to the increased amount; and
automatically deliver insulin to the user with the pump mechanism according to the modified basal rate.

16. The system of claim 15, wherein the at least one processor is further configured to determine that the user's glucose levels are increasing at greater than a predetermined rate and the at least one processor is configured to provide a further increase to the amount by which the limiter function will permit the basal rate to be increased if the user's glucose levels are increasing at greater than the predetermined rate.

17. The system of claim 15, wherein the high glucose threshold is a different glucose level than the glucose target.

18. The system of claim 17, wherein the high glucose threshold is a higher glucose level than the glucose target.

19. The system of claim 15, wherein the glucose target used by the closed loop algorithm is a single glucose value.

20. The system of claim 15, wherein the glucose target used by the closed loop algorithm is a target glucose range.
